(19) United States
(12) Patent Application Publication
(10) Pub. No.: US 2025/0249130 A1
(43) Pub. Date: Aug. 7, 2025

(54) RADIOPAQUE POLYPEPTIDES FOR INJECTABLE COMPOSITIONS

(71) Applicant: Boston Scientific Scimed, Inc., Maple Grove, MN (US)

(72) Inventors: Cristian Parisi, Amherst, MA (US); Nicolas Ball-Jones, Minneapolis, MN (US); Yen-Hao Hsu, Shrewsbury, MA (US); Rick Dorn, Plymouth, MN (US); Joseph Thomas Delaney, JR., Minneapolis, MN (US)

(73) Assignee: Boston Scientific Scimed, Inc., Maple Grove, MN (US)

(21) Appl. No.: 19/045,850

(22) Filed: Feb. 5, 2025

Related U.S. Application Data
(60) Provisional application No. 63/550,422, filed on Feb. 6, 2024.

(51) Int. Cl. A61K 49/04; A61K 9/00; A61K 47/02

(52) U.S. Cl. CPC A61K 49/0438; A61K 9/0019; A61K 47/02

(57) ABSTRACT

In some aspects, the present disclosure pertains to injectable compositions that comprise (a) iodinated polypeptide-containing molecules, (b) silicate microparticles, and (c) water. In other aspects, the present disclosure pertains to kits that comprise one or more containers that contain such an injectable composition and a delivery device. In still other aspects, the present disclosure pertains to medical procedures that comprise administering such an injectable composition to a subject.

### CROSS-REFERENCE TO RELATED APPLICATIONS

The application claims the benefit of U.S. Provisional Patent Application Ser. No. 63/550,422 filed on Feb. 6, 2024, the disclosure of which is incorporated herein by reference.

### FIELD

The present disclosure relates to injectable compositions in which radiopacity is introduced via iodinated polypeptides. The injectable compositions are useful in various medical procedures.

### BACKGROUND

Injectable shear-thinning compositions are attractive due to their minimally invasive delivery procedure, providing reduced healing time, reduced scarring, decreased risk of infection, and ease of delivery compared with surgically implanted materials. Injectable shear-thinning compositions are especially useful for applications where the final form and shape are either not important or are defined by the void or space into which they are injected. Due to their ease of delivery, injectable shear-thinning compositions are potentially useful in a number of areas such as providing a structural or space-filling function, functioning as embolic agents for diverting or eliminating flow in blood vessels, acting as tissue engineering compositions, and delivery of drugs, among other medical applications.

One currently available injectable shear-thinning composition is Obsidio™ Conformable Embolic (Boston Scientific Corporation, Marlborough, Massachusetts, USA). It is pre-packaged in a ready-to-use syringe. As the material is pushed through a catheter on its way to a targeted site during administration, the material shear-thins and flows readily, like a liquid. When shear forces are removed as the material reaches its intended location, it reverts to a soft solid that molds to the targeted vasculature's shape, creating a physical barrier that stops the blood flow. The Obsidio™ embolic composition contains laponite, gelatin, water and tantalum and is indicated to control bleeding and stop blood flow to tumors in the peripheral vasculature. The Obsidio™ material is radiopaque due to the presence of tantalum particles in its formulation. Every Obsidio™ material component is resorbable, but the tantalum itself. Some applications, such as, but not limited to, spacers for prostate cancer radiotherapy (or radiotherapy for other organs/tissues), require a full absorption of all components. This issue can be resolved by removing tantalum from Obsidio™ formulation. However, this results in reduced radiopacity.

### SUMMARY

There is an ongoing need in the biomedical arts for injectable compositions that are fully absorbable, while at the same time have sufficient radiopacity for enhanced imaging during and after injection.

In some aspects, the present disclosure pertains to injectable compositions that comprise (a) iodinated polypeptide-containing molecules, (b) silicate microparticles, and (c) water.

In some embodiments, the iodinated polypeptide-containing molecules are iodinated collagen-based proteins.

In some embodiments, which can be used in conjunction with the above aspects and embodiments, the iodinated polypeptide-containing molecules comprise iodinated gelatin molecules.

In some embodiments, which can be used in conjunction with the above aspects and embodiments, the silicate microparticles comprise natural and/or synthetic silicate layered clays.

In some embodiments, which can be used in conjunction with the above aspects and embodiments, the injectable composition is an injectable shear-thinning composition.

In some embodiments, which can be used in conjunction with the above aspects and embodiments, the iodinated polypeptide-containing molecules comprise covalently attached iodinated moieties. In some embodiments, the iodinated polypeptide-containing molecules are formed by reacting amine groups of non-iodinated polypeptide-containing molecules with activated-ester-substituted iodinated molecules in an amide coupling process. In some embodiments, the iodinated polypeptide-containing molecules are formed by (a) esterifying carboxyl groups of non-iodinated polypeptide-containing molecules to form esterified non-iodinated polypeptide-containing molecules and (b) reacting amine groups of the esterified non-iodinated polypeptide-containing molecules with carboxyl-substituted iodinated molecules in an amide coupling process. In some embodiments, the iodinated polypeptide-containing molecules are de-esterified after the amide coupling process. In some embodiments, the iodinated polypeptide-containing molecules are formed by reacting carboxyl groups of non-iodinated polypeptide-containing molecules with diazo-substituted iodinated molecules in an ester coupling process.

In some embodiments, which can be used in conjunction with the above aspects and embodiments, the iodinated polypeptide-containing molecules comprise ionically coordinated carboxyl-substituted iodinated molecules.

In some embodiments, which can be used in conjunction with the above aspects and embodiments, the injectable composition further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the injectable composition is a sterile composition.

In some embodiments, which can be used in conjunction with the above aspects and embodiments, the injectable composition is provided in a preloaded syringe.

In other aspects, the present disclosure pertains to kits that comprise one or more containers that contain an injectable composition in accordance with the above aspects and embodiments, and a delivery device. In some embodiments, the delivery device comprises a syringe, a needle and optionally, a catheter.

In other aspects, the present disclosure pertains to medical procedures that comprise administering an injectable composition in accordance with the above aspects and embodiments to a subject.

In some embodiments, the method comprises injecting the injectable composition into the subject. In some of these embodiments, the administering comprises parenteral administration.

In some embodiments, which can be used in conjunction with the above aspects and embodiments, the administering is performed using a catheter and/or a syringe.

In some embodiments, which can be used in conjunction with the above aspects and embodiments, the administering is performed under image guidance.

The above and other aspects, embodiments, features and benefits of the present disclosure will be readily apparent from the following detailed description.

### BRIEF DESCRIPTION OF THE DRAWINGS

FIG. 1A schematically illustrates a process for forming an activated-ester-substituted iodinated molecule from a carboxyl-functional iodinated molecule, in accordance with an embodiment of the present disclosure.

FIG. 1B schematically illustrates a process for forming an iodinated polypeptide-containing molecule from an activated-ester-substituted iodinated molecule and a non-iodinated polypeptide-containing molecule, in accordance with an embodiment of the present disclosure.

FIG. 2 schematically illustrates a process for forming an activated-ester-substituted iodinated molecule from an amino-functional iodinated molecule, in accordance with an embodiment of the present disclosure.

FIG. 3 schematically illustrates a process for forming an activated-ester-substituted iodinated molecule from an amino-functional iodinated molecule, in accordance with another embodiment of the present disclosure.

FIG. 4 schematically illustrates ionic coordination between a polypeptide-containing molecule and a carboxyl-functional iodinated molecule, in accordance with an embodiment of the present disclosure.

FIGS. 5A-5B schematically illustrate a process for forming an iodinated polypeptide-containing molecule from a carboxyl-functional iodinated molecule and a non-iodinated polypeptide-containing molecule, in accordance with an embodiment of the present disclosure.

FIG. 6 schematically illustrates a process for forming an iodinated polypeptide-containing molecule from a diazo-substituted iodinated molecule and a non-iodinated polypeptide-containing molecule, in accordance with an embodiment of the present disclosure.

FIG. 7 schematically illustrates a catheter and a syringe that is loaded with an injectable shear-thinning composition, in accordance with an embodiment of the present disclosure.

### DETAILED DESCRIPTION

In various aspects, the present disclosure relates to radiopaque polypeptide-containing molecules, which can be used, for example, in injectable compositions.

In some embodiments, the injectable compositions are injectable shear-thinning compositions. The phrase “shear-thinning” refers to a decrease in viscosity of a composition with increasing application of shear stress. A shear-thinning composition (i.e. a composition exhibiting shear-thinning behavior) can exhibit a decrease in viscosity (i.e. increase in flow) upon application of an increasing rate of shear stress.

In some embodiments, the radiopaque polypeptide-containing molecules are radiopaque fibrous protein molecules. Fibrous proteins are made up of polypeptide chains that are elongated and fibrous in nature or have a sheet-like structure. Fibrous proteins for use in the injectable compositions of the present disclosure include animal-derived proteins and non-animal-derived proteins. Fibrous proteins for use in the injectable compositions of the present disclosure include keratin, elastin, fibroin, myosin, desmin, fibrin, actin, and collagen, including denatured and hydrolyzed forms thereof such as gelatin, polyarginine, polylysine, and spider silk proteins. Specific fibrous proteins for use herein include porcine gelatin (e.g., type-A porcine gelatin, gelatin derived from porcine skin, gelatin derived from porcine bones, and the like), bovine gelatin (e.g., type-B bovine gelatin, gelatin derived from bovine skin, gelatin derived from bovine bones, and the like), equine gelatin, avian-derived gelatin and fish-derived gelatin.

In the present disclosure, iodinated polypeptide-containing molecules can be formed by functionalizing non-iodinated polypeptide-containing molecules. The polypeptide-containing molecules may be functionalized by covalently attaching molecules that comprise iodinated moieties to the polypeptide-containing molecules. Iodinated moieties can comprise one, two, three, four, five, six, seven, eight, nine, ten, eleven, twelve or more iodine atoms. In various embodiments, the iodinated moieties comprise one or more iodinated aromatic groups.

Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six, or more iodine atoms. In some of these embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups, hydroxyalkyl groups (e.g., hydroxyalkyl groups containing one hydroxyl group, two hydroxyl groups, three hydroxyl groups, four hydroxyl groups, etc. and one carbon, two carbons, three carbons, four carbons, etc.). The hydrophilic groups may be amide groups, ester groups, amine groups, and ether groups, among others. The hydrophilic groups may be linked to the aromatic groups directly or through any suitable linking moiety, which may be selected, for example, from alkyl groups, ether groups, ester groups, amide groups, amine groups, carbonate groups, and combinations thereof, among others.

In various aspects, non-iodinated polypeptide-containing molecules are functionalized through the use of carboxyl-functional iodinated molecules. In these embodiments, iodinated polypeptide-containing molecules may be formed by methods that comprise coupling a carboxyl-functional iodinated molecule with amino acid residues in a polypeptide-containing molecule that have amine-containing side groups (e.g., amino acid residues containing —NH₂ groups, including C₁-C₆-aminoalkyl and guanidino groups), for example, lysine, arginine, and/or ornithine amino acid residues of a polypeptide-containing molecule, in an amide coupling reaction. Carboxyl-functional iodinated molecules for use herein include carboxyl-functional iodinated molecules that comprise one or more iodinated aromatic groups, some examples of which are described above, among others.

In some embodiments, to enhance selectivity, carboxyl group(s) of the carboxyl-functional iodinated molecule are converted into activated ester groups before coupling with the polypeptide-containing molecule. Examples of activated ester groups include cyclic imide ester groups, such as succinimide ester groups, maleimide ester groups, glutarimide ester groups, diglycolimide ester groups, phthalimide ester groups, and bicyclo[2.2.1]hept-5-ene-2,3-dicarboxylic acid imide ester groups, among other possibilities.

An activated ester group may be formed at the site of the carboxyl group(s) of the carboxyl-functional iodinated molecule, for example, by reacting an N-hydroxy cyclic imide molecule (e.g., N-hydroxysuccinimide, N-hydroxymaleimide, N-hydroxyglutarimide, N-hydroxyphthalimide, N-hydroxy-5-norbornene-2,3-dicarboxylic acid imide, also known as N-hydroxybicyclo[2.2.1]hept-5-ene-2,3-dicarboxylic acid imide (HONB), etc.) with the carboxyl group(s) of the carboxyl-functional iodinated molecule in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester group (e.g., a succinimide ester group, a maleimide ester group, a glutarimide ester group, a phthalimide ester group, a bicyclo[2.2.1]hept-5-ene-2,3-dicarboxylic acid imide ester group, etc.). In this way, a variety activated ester groups can be formed.

Carboxyl-functional iodinated molecules for use in the present disclosure include triiodobenzoic acid

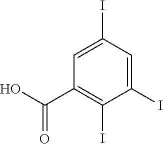 [SMILES: O=C(O)c1cc(I)cc(I)c1I]

(CAS #88-82-4), diatrizoic acid,

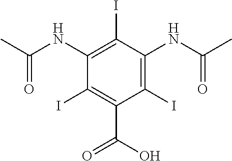 [SMILES: CC(=O)Nc1c(I)c(NC(C)=O)c(I)c(C(=O)O)c1I]

(CAS #117-96-4), N-acetyl-3,5-diiodo-L-tyrosine,

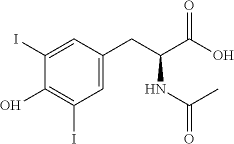 [SMILES: CC(=O)N[C@@H](Cc1cc(I)c(O)c(I)c1)C(=O)O]

(CAS #1027-28-7), N-acetyl-3-diiodo-L-tyrosine,

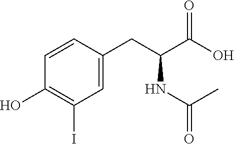 [SMILES: CC(=O)N[C@@H](Cc1ccc(O)c(I)c1)C(=O)O]

(CAS #1023-47-8) and N-acetyl-thyroxine,

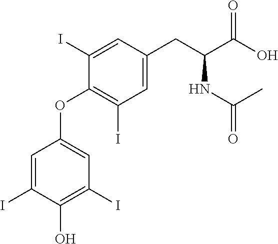 [SMILES: CC(=O)N[C@@H](Cc1cc(I)c(Oc2cc(I)c(O)c(I)c2)c(I)c1)C(=O)O]

(CAS #26041-51-0), among many others.

Additional carboxyl-functional iodinated molecules can be found in the following table.

| | | # I |
|---|---|---|
| Name | CAS# | atoms |
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoic acid | 117-96-4 | 3 |
| 2,3,5-Triiodobenzoic acid | 88-82-4 | 3 |
| Acetrizoic acid | 85-36-9 | 3 |
| Metrizoic acid | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid* | 79211-41-9 | 3 |
| 2,4,6-Triiodobenzoic acid | 2012-31-9 | 3 |
| Benzoic acid, 3-[[[2-hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-, (S)- | | |
| (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047-48-8 | 3 |
| triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid* | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid* | 2618-25-9 | 6 |
| Iocarmic acid* | 10397-75-8 | 6 |

*molecules having more than one carboxyl group

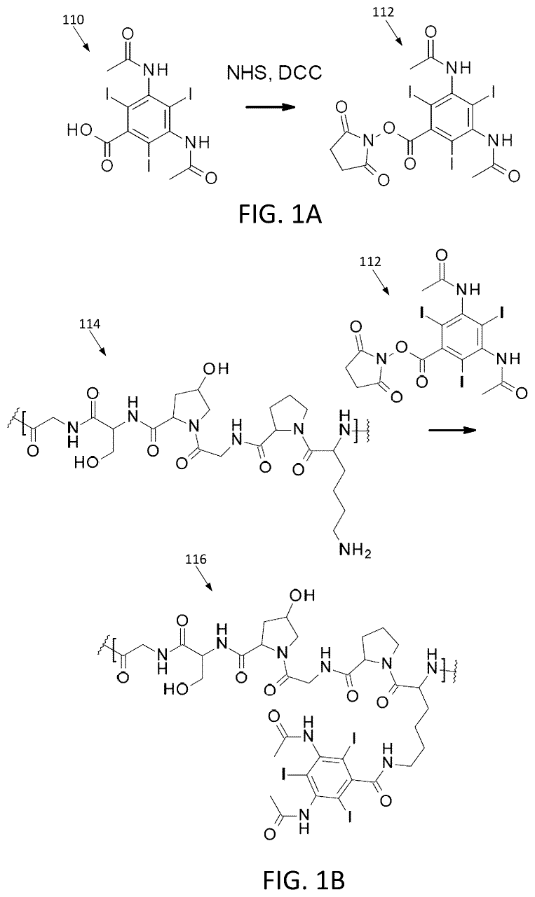

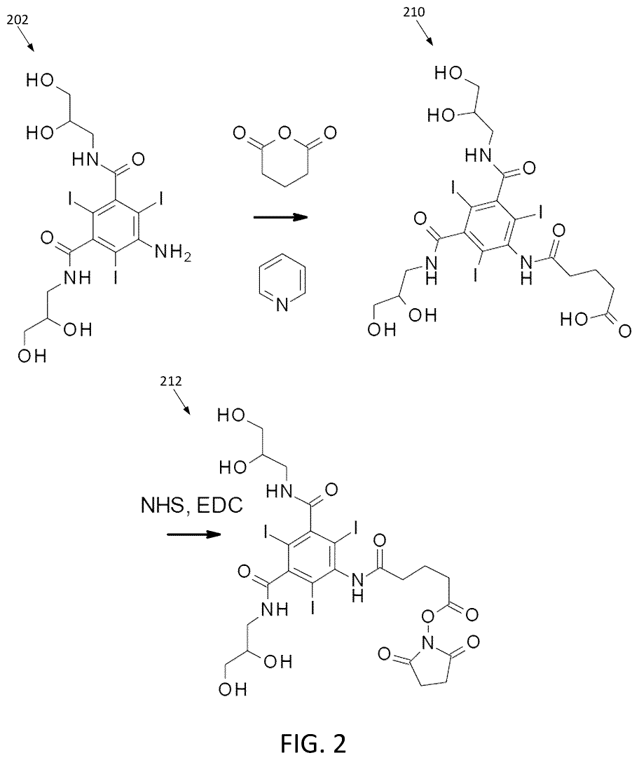

As a specific example, and with reference to FIG. 1A, in a first step, the carboxylic acid group of a carboxyl-functional iodinated molecule, specifically, diatrizoic acid (110), is reacted with an N-hydroxy cyclic imide molecule, specifically, N-hydroxysuccinimide (NHS), in the presence of a suitable ester coupling agent, specifically, N,N′-dicyclohexylcarbodiimide (DCC), to form an activated-ester-substituted iodinated molecule, specifically, a cyclic-imide-ester-substituted iodinated molecule, more specifically, an N-succinimidyl-ester-substituted molecule, i.e., N-succinimidyl-2,4,6-triiodo-3,5-diacetamidobenzoate (112).

Then, in a subsequent step as shown in FIG. 1B, the N-succinimidyl-2,4,6-triiodo-3,5-diacetamidobenzoate (112) is reacted in an amide coupling reaction with amine-containing side groups of amino acid residues of a polypeptide-containing molecule, specifically, a C₄-aminoalkyl side group of a lysine residue within a sequence of a fibrous protein molecule, in particular, gelatin (114), with the result being an iodinated fibrous protein molecule, specifically, iodinated gelatin (116) in which a 2,4,6-triiodo-3,5-diacetamidophenyl group is linked to the lysine residue through an amide linkage.

By performing the reaction with a molar excess of activated-ester-substituted iodinated molecules relative to the total number of moles of amine groups provided by the polypeptide-containing molecule in the reaction mixture, a single activated-ester-substituted iodinated molecule will be reacted with each of the available amine groups of the polypeptide-containing molecule. On the other hand, by performing the reaction with a molar excess of amine groups relative to activated-ester-substituted iodinated molecules, some of the available amine groups will remain unreacted in the resulting iodinated polypeptide-containing molecule. For example, some of the aminoalkyl side groups of the lysine residues of the iodinated polypeptide-containing molecule may remain unreacted, as may the amine group at the N-terminus of the iodinated polypeptide-containing molecule (not illustrated in FIG. 1B).

In various embodiments, it may be desirable to form a carboxyl-functional iodinated molecule from another iodinated molecule that is lacking a carboxyl group. For example, in some embodiments, an amine-functional iodinated molecule may be reacted with a cyclic anhydride in a ring opening reaction, thereby forming a carboxyl-terminated pendant group at the position previously occupied by the amine group of the amine-functional iodinated molecule.

Examples of amine-functional iodinated molecules include 5-amino-N,N′-bis(2,3-dihydroxypropyl)-2,4,6-triiodoisophthalamide (also known as iohexol related compound J),

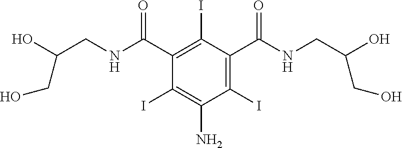 [SMILES: Nc1c(I)c(C(=O)NCC(O)CO)c(I)c(C(=O)NCC(O)CO)c1I]

(CAS #CAS 76801-93-9), acetal-protected 5-amino-N,N′-bis(2,3-dihydroxypropyl)-2,4,6-triiodoisophthalamide (acetal-protected iohexol related compound J),

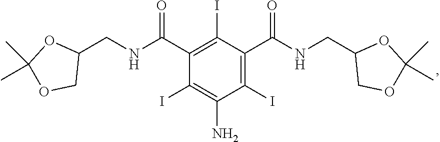

5-amino-N,N′-bis(2,3-dihydroxypropyl)-2,4,6-triiodoisophthalamide (also known as Iohexol EP Impurity F),

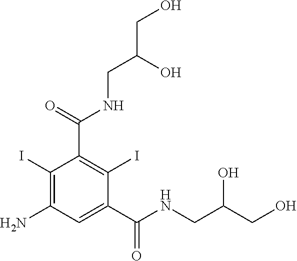 [SMILES: Nc1cc(C(=O)NCC(O)CO)c(I)c(C(=O)NCC(O)CO)c1I]

(CAS #1215856-35-1), acetal-protected Iohexol EP Impurity F, dimethyl 5-amino-2,4,6-triiodo-1,3-benzenedicarboxylate,

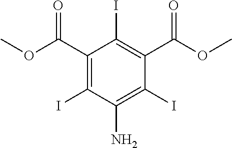

(CAS #154921-11-6), 2,4,6-triiodoanaline,

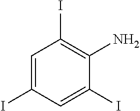

(CAS #24154-37-8), and triiodobenzylamine,

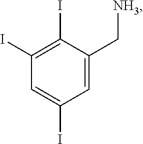

among others.

Cyclic anhydrides include, for example, succinic anhydride,

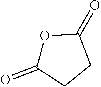

(CAS #108-30-5), glutaric anhydride,

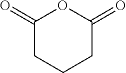

(CAS #108-55-4), and diglycolic anhydride,

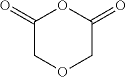

(CAS #4480-83-5). Additional anhydrides, diglycolic anhydride, some of which are iodinated, are shown in the following table.

| Name | CAS # |
|---|---|
| Adipic Anhydride | 2035-75-8 |
| 2,8-Oxocanedione | 10521-07-0 |
| 3-Oxabicyclo[3.1.0]hexane-2,4-dione | 5617-74-3 |
| 3-Oxabicyclo[3.2.0]heptane-2,4-dione | 4462-96-8 |
| Dihydro-4-methyl-2H-pyran-2,6(3H)-dione | 4166-53-4 |
| Itaconic anhydride | 2170-03-8 |
| 2,2-Dimethylsuccinic anhydride | 17347-61-4 |
| 6,6-Dimethyl-3-oxabicyclo[3.1.0]hexane-2,4-dione | 5466-84-2 |
| Dihydro-4,4-dimethyl-2H-pyran-2,6(3H)-dione | 4160-82-1 |
| 2H-Pyran-2,4,6(3H,5H)-trione | 10521-08-1 |
| Dihydro-4-(2-methylpropyl)-2H-pyran-2,6(3H)-dione | 185815-59-2 |
| 3-Iodophthalic anhydride* | 28418-88-4 |
| Tetraiodophthalic anhydride* | 632-80-4 |
| 5-Iodo-1,3-isobenzofurandione* | 28418-89-5 |
| 5,6-Diiodo-1,3-isobenzofurandione* | 25834-17-7 |
| Dihydro-3-(2-iodophenyl)-2,5-furandione* | 887131-98-8 |

*Iodinated

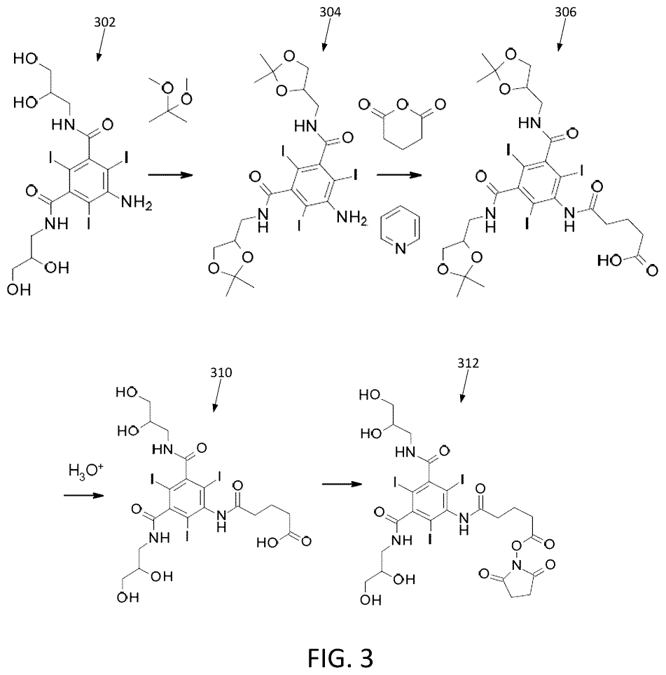

As a specific example, and with reference to FIG. 2, in a first step, a primary amine group of an amino-functional iodinated molecule, specifically, iohexol related compound J (202), also known as 5-amino-N,N′-bis(2,3-dihydroxypropyl)-2,4,6-triiodo-1,3-benzenedicarboxamide, is reacted in a ring opening reaction with a cyclic anhydride, specifically, glutaric anhydride, optionally with an added base (in a catalytic amount or stochiometric, depending on the base) to help promote the ring opening reaction, thereby forming a carboxyalkylcarbonylamino pendant group, specifically, a carboxypropylcarbonylamino group at the position previously occupied by the amine group of the amine-functional iodinated molecule. Then, the carboxylic acid group of the resulting carboxyl-functional iodinated molecule, specifically, 5-carboxypropylcarbonylamino-N,N′-bis(2,3-dihydroxypropyl)-2,4,6-triiodo-1,3-benzenedicarboxamide (210), is reacted with an N-hydroxy cyclic imide molecule, specifically, N-hydroxysuccinimide (NHS), in the presence of a suitable ester coupling agent, specifically, N,N′-dicyclohexylcarbodiimide (DCC), to form an activated-ester-substituted iodinated molecule, specifically, a cyclic-imide-ester-substituted iodinated molecule, more specifically, a succinimidyl-ester-substituted molecule, more specifically, succinimdyl glutarate functionalized iohexol (212). This molecule can then be reacted in an amide coupling reaction with amine-containing side groups of amino acid residues of a polypeptide-containing molecule along the lines shown in FIG. 1B.

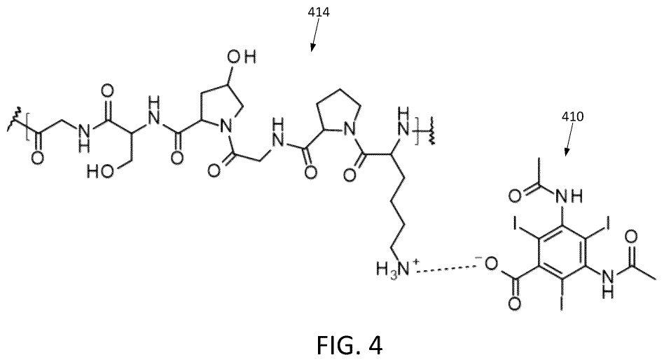

In an alternative strategy shown in FIG. 3, in a first step, adjacent hydroxyl groups of iohexol related compound J (302) are protected using 2,2-dimethoxypropane in the presence of p-toluenesulfonic acid at room temperature, to obtain an acetal-protected hydroxy-functional iodinated molecule, specifically, acetal-protected iohexol related compound J (304). Then, analogous to FIG. 2, the primary amine group of the acetal-protected iohexol related compound J (304) is reacted in a ring opening reaction with a cyclic anhydride, specifically, glutaric anhydride, optionally with an added base to help promote the ring opening reaction, to form carboxypropylcarbonylamino group at the position previously occupied by the amine group of the amine-functional iodinated molecule. Deprotection of the resulting acetal-protected molecule (306) in acid yields a carboxyl-functional iodinated molecule, specifically, 5-carboxypropylcarbonylamino-N,N′-bis(2,3-dihydroxypropyl)-2,4,6-triiodo-1,3-benzenedicarboxamide (310), which is reacted with an N-hydroxy cyclic imide molecule, specifically, N-hydroxysuccinimide (NHS), in the presence of a suitable ester coupling agent to form an activated-ester-substituted iodinated molecule (312). This molecule can then be reacted in an amide coupling reaction with amine-containing side groups of amino acid residues of a polypeptide-containing molecule along the lines shown in FIG. 1B.

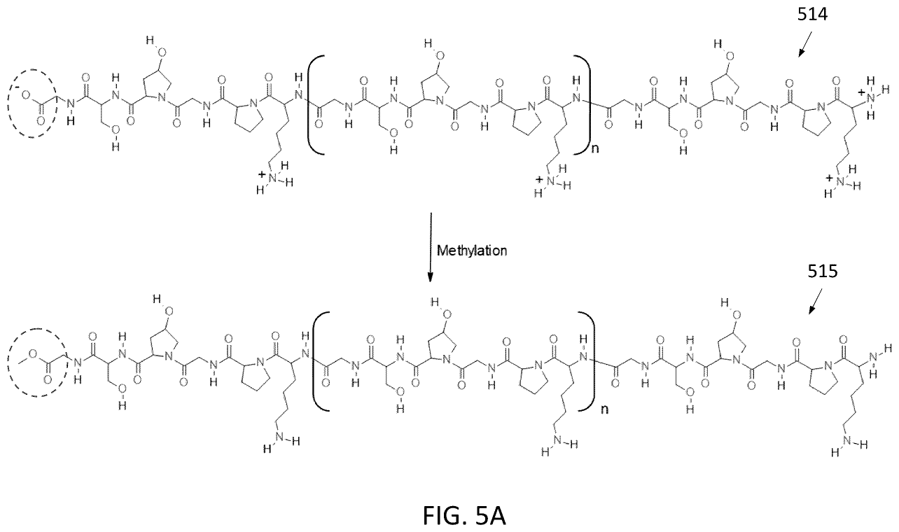

Although covalent functionalization of amine-containing side groups of amino acid residues of polypeptide-containing molecules with carboxyl-functional iodinated molecules are described above, in other embodiments the carboxyl-functional iodinated molecules can be ionically coordinated with the amine-containing side groups to form an iodinated polypeptide-containing molecule. As a specific example, and with reference to FIG. 4, a negatively charged carboxyl group of diatrizoic acid (410) is shown ionically coordinated with a positively charged amine-containing side group of an amino acid residue of a polypeptide-containing molecule, specifically, a positively charged C₄-aminoalkyl side group of a lysine residue within a sequence of a fibrous protein molecule, in particular, gelatin (414), The environment of FIG. 4 has a relatively neutral pH at which the carboxyl groups of the carboxyl-functional iodinated molecule and the amine groups of the polypeptide-containing molecule are both charged.

In the preceding embodiments, the carboxyl group(s) of the carboxyl-functional iodinated molecule are converted into activated ester groups before coupling with the polypeptide-containing molecule. In other embodiments, unmodified carboxyl group(s) of the carboxyl-functional iodinated molecule are directly reacted with the amine-containing side groups, for example, the amine-containing side groups of lysine, arginine, and/or ornithine amino acid residues, of a polypeptide-containing molecule in an amide coupling reaction. However, polypeptide-containing molecules also generally contain carboxyl groups, for example, a carboxylic acid terminal group and/or carboxyl-containing side groups (e.g., carboxyl-containing side groups of glutamic acid or aspartic acid residues), which can react with the amine groups in the polypeptide-containing molecule. To prevent this, the carboxyl groups of the polypeptide-containing molecule can be esterified, for example, by methylation of the carboxyl groups.

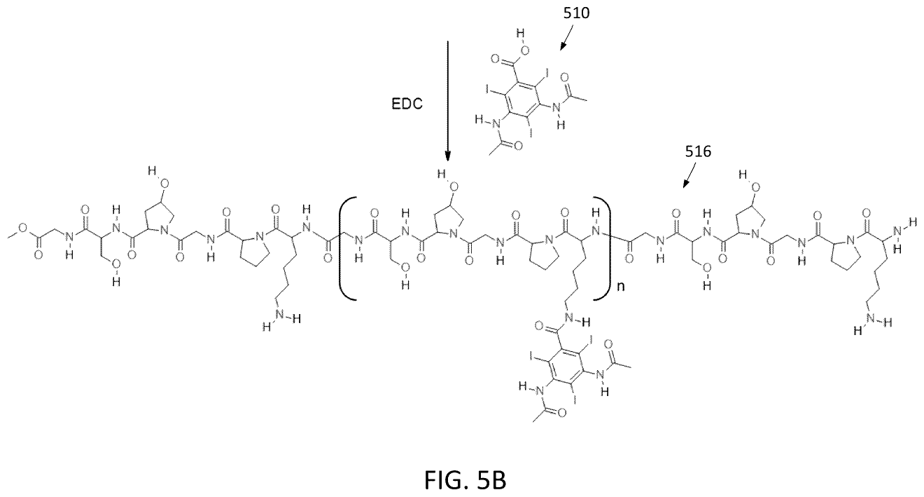

For example, turning now to FIG. 5A, a carboxyl group at the C-terminus of a polypeptide-containing molecule (514) (highlighted with a dashed oval) may be subjected to a methylation procedure to form a methylated polypeptide-containing molecule (515) wherein is methoxycarbonyl group (also highlighted with a dashed oval) is formed at the site of the carboxyl group of the polypeptide-containing molecule (514), in which n is an integer of 1 or more. In a typical procedure, methylation can be performed by reaction with diazomethane or trimethylsilyldiazomethane. Although the polypeptide-containing molecule 514 shown in FIG. 5A does not have any carboxyl side groups, if such groups had been present, they would have been methylated as well, so long as an excess of the methylation agent is present during the reaction.

Subsequently, as shown in FIG. 5B, an amide coupling reaction can be performed between a carboxyl group of a carboxyl-functional iodinated molecule, specifically, diatrizoic acid (510), and an amine group of the methylated polypeptide-containing molecule (515), with the result being an iodinated methylated polypeptide-containing molecule (516) in which a 2,4,6-triiodo-3,5-diacetamidophenyl group is linked to lysine residues through an amide linkage.

By performing the reaction with a molar excess of the number of carboxyl-functional iodinated molecules relative to the total number of moles of amine groups provided by the methylated polypeptide-containing molecule in the reaction mixture, a single carboxyl-functional iodinated molecule will be reacted with each of the available amine groups of the methylated polypeptide-containing molecule. On the other hand, by performing the reaction with a molar excess of amine groups relative to carboxyl-functional iodinated molecules, some of the available amine groups will remain unreacted in the resulting iodinated methylated polypeptide-containing molecule. For example, as shown in FIG. 5B some of the aminoalkyl side groups of the lysine residues of the iodinated methylated polypeptide molecule (516) remain unreacted, as does the amine group at the N-terminus of the iodinated methylated polypeptide molecule (516).

In cases where carboxyl-functional iodinated molecules are employed that have more than one carboxyl group (including those designated with an asterisk (*) in the above table of carboxyl-functional iodinated molecules), a significant excess of the iodinated species is preferably used during the coupling reaction to ensure that the carboxyl-functional iodinated molecules attach to only a single methylated polypeptide-containing molecule. The product may be further purified via chromatography methods or fractional crystallization.

In some embodiments, the iodinated methylated polypeptide molecule is then de-esterified to restore the carboxyl groups of the original polypeptide-containing molecule, for example, by incubating in water at pH of 7, or subjection of proteins to reaction with carboxyl methylesterases (see, e.g., Jack R. Barber and Steven Clarke, “Demethylation of protein carboxyl methyl esters: a nonenzymatic process in human erythrocytes?” Biochemistry 1985, 24, 18, 4867-4871).

In other aspects, the present disclosure pertains to methods of forming iodinated polypeptide-containing molecules that comprise coupling a diazo-functional iodinated molecule with amino acid residues of the polypeptide-containing molecule that have carboxyl-containing side groups (e.g., glutamic acid and/or aspartic acid residues) in an ester coupling reaction. Diazo-functional iodinated molecules for use herein include diazo-functional iodinated molecules that comprise one or more iodinated aromatic groups, some examples of which are described above.

Diazo-functional iodinated molecules for use in the present disclosure include diazo-functional iodinated aromatic molecules such as a-aryl-a-diazoamide molecules, where the aryl group is an iodinated aryl group. These molecules can be synthesized by reaction with superstoichiometric amounts of aromatic molecules containing two to six iodine functional groups as outlined above through palladium catalyzed reaction with N-succinimidyl 2-diazoacetate. This reaction will consume one of the aryl iodide functional groups to append the radiopaque moiety to the diazo compound. A superstoichiometric amount of aryl iodide molecules is necessary to prevent multiple appending events on the aryl iodide. Afterwards reaction with the chosen amine displaces the succinimide functional group (see, e.g., Joomyung V. Jun, Ronald T. Raines, “Two-Step Synthesis of α-Aryl-α-diazoamides as Modular Bioreversible Labels”, Org Lett. 2021 Apr. 16; 23 (8): 3110-3114.)

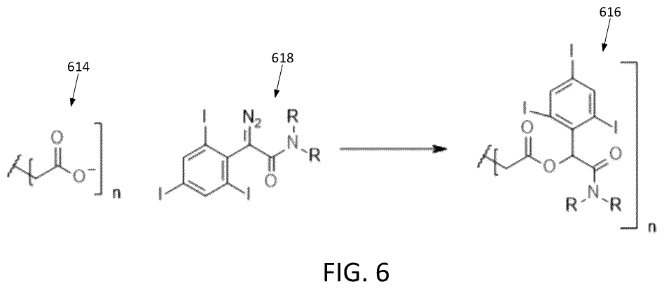

As a specific example, and with reference to FIG. 6, a diazo-functional iodinated aromatic molecule, specifically, an α-iodoaryl-α-diazoamide (618) in which

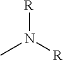

is a 1-pyrrolidinyl group

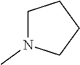

or any amine containing functional group, is reacted in an ester coupling reaction in an aqueous environment with carboxyl-containing side groups of amino acid residues of a polypeptide-containing molecule (614), where n represents the number of carboxyl groups that are reacted, with the result being an iodinated polypeptide-containing molecule (616), in which a 2,4,6-triiodophenyl group is linked to the amino acid resides through an ester linkage.

By performing the reaction with a molar excess of diazo-functional iodinated molecule relative to the total number of moles of carboxyl groups provided by the polypeptide-containing molecule in the reaction mixture, a single diazo-functional iodinated molecule will be reacted with each of the available carboxyl groups of the polypeptide-containing molecule. On the other hand, by performing the reaction with a molar excess of carboxyl groups relative to diazo-functional iodinated molecules, some of the available carboxyl groups will remain unreacted in the resulting iodinated polypeptide-containing molecule. For example, carboxyl-containing side groups of glutamic acid and/or aspartic acid residues of the iodinated polypeptide-containing molecule may remain unreacted, as may the carboxyl group at the C-terminus of the iodinated polypeptide-containing molecule.

In some aspects, the present disclosure pertains to injectable compositions that comprise (a) one or more types of iodinated polypeptide-containing molecules, such as those described above, (b) one or more types of silicate microparticles, (c) water. In various embodiments, the injectable compositions are injectable shear-thinning compositions.

In some embodiments, the injectable compositions of the present disclosure have a pH between 6 and 11, more typically between 8 and 10.

In some embodiments, the injectable compositions of the present disclosure are physically crosslinked hydrogel compositions.

In some embodiments, the injectable compositions of the present disclosure are ionically crosslinked hydrogel compositions.

As used herein, a “hydrogel” refers to a hydrated, three-dimensional polymer-containing network.

In some embodiments, the injectable compositions of the present disclosure flow upon application of a pressure greater than a yield stress of the injectable compositions.

In some embodiments, the injectable compositions of the present disclosure contain between 0.05 wt % or less or less and 30 wt % or more of one or more types of polypeptide-containing molecule, for example, ranging anywhere from 0.05 wt % to 0.10 wt % to 0.3 wt % to 0.5 wt % to 1 wt % to 3 wt % to 10 wt % to 30 wt % (i.e., ranging between any two of the preceding values) of one or more types of polypeptide-containing molecule, typically, ranging between 0.5 wt % and 10 wt % of one or more types of polypeptide-containing molecule.

Silicate microparticles for use in the injectable compositions of the present disclosure include natural silicate microparticles and synthetic silicate microparticles. Particular examples of silicate microparticles include natural and synthetic silicate layered clays. Natural silicate layered clays include montmorillonite, saponite, hectorite, kaolinite, palygorskite and sepiolite, among others. Synthetic silicate layered clays include lithium magnesium sodium silicates such as Laponite®-based silicate nanoplatelets (e.g., Laponite® XLG-based silicate nanoplatelets, Laponite® XLS-based silicate nanoplatelets, Laponite® XL21-based silicate nanoplatelets, and Laponite® D-based silicate nanoplatelets), Sumecton® SWN and Lucentite™ SWN, magnesium aluminum silicates such as Sumecton® SA, sodium magnesium silicates such as Optigel® SH and SUPLITE-MP, and fluoromica such as Somasif™ ME100, among others.

Silicate microparticles for use in the injectable compositions of the present disclosure may have a size ranging from 5 nm or less to 75 nm or more in longest dimension (e.g., diameter for a sphere, length for a rod, greatest width for a plate-shaped particle, etc.), for example ranging anywhere from 5 nm to 10 nm to 25 nm to 50 nm to 75 nm in longest dimension.

The silicate microparticles for use in the injectable compositions of the present disclosure include microparticles that have a neutral charge, microparticles that have a net positive charge, and microparticles that have a net negative charge. The net charge of the silicate microparticles may depend upon the pH of the injectable composition. In some embodiments, the silicate microparticles have a net positive charge at the pH of the of the injectable composition. In some embodiments, the silicate microparticles have a negative positive charge at the pH of the of the injectable composition.

In some embodiments, the silicate microparticles are plate-shaped. In some embodiments, the silicate microparticles are silicate layered clays characterized by a discotic charge distribution on the surface. In some embodiments, the plate-shaped silicate microparticles comprise a positively charged edge and a negatively charged surface. In some embodiments, the overall charge of the silicate microparticles is negative. In some embodiments, the plate-shaped silicate microparticles are from about 5 nm to about 60 nm in diameter, for example, from about 10 nm to about 40 nm in diameter, from about 10 nm to about 30 nm in diameter, or from about 20 to about 30 nm in diameter. In some embodiments, the plate-shaped silicate microparticles are from about 0.5 nm to about 2 nm in thickness, or about 1 nm in thickness.

In some embodiments, the injectable compositions of the present disclosure contain between 1 wt % and 15 wt % of one or more types of silicate microparticles, typically between 2 wt % and 8 wt %.

Silicate microparticles for use in the injectable compositions of the present disclosure may have a size ranging from 5 nm to 70 microns in longest dimension (e.g., diameter for a sphere, length for a rod, greatest width for a plate-shaped particle, etc.), for example, ranging anywhere from 5 nm to 10 nm to 25 nm to 50 to 100 nm to 250 nm to 500 nm to 1 micron to 2.5 microns to 5 microns to 10 microns to 50 microns to 70 microns.

In various embodiments, the injectable compositions in accordance with the present disclosure have a radiopacity that is greater than 100 Hounsfield units (HU), beneficially ranging anywhere from 100 HU to 250 HU to 500 HU to 750 HU to 1000 HU or more, for example, when measured on bench-top micro-CT systems such as Xtreme CT from Scanco Medical (Wangen-Brüttisellen, Switzerland) or similar.

The water in the injectable compositions of the present disclosure may be provided in the form of ultrapure water, water for injection, saline, phosphate buffered saline, or high-ion-content water.

In some embodiments, the injectable compositions of the present disclosure contain between 40 wt % and 99 wt % of water, for example, ranging anywhere from 40 wt % to 50 wt % to 65 wt % to 80 wt % to 90 wt % to 95 wt % of water, typically, between 65 wt % and 95 wt % of water.

The injectable compositions of the present disclosure may be formed using a variety of methods. The one or more types of iodinated polypeptide-containing molecules, one or more types of silicate microparticles, and water may be mixed in any suitable order. Mixing may be performed by any suitable mixing technique, including, for example, centrifugal mixing, manual mixing, high shear dispersing, vacuum mixing, vortexing, and/or syringe-to-syringe mixing.

The injectable compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the injectable compositions may be sterilized via sterile filtration and/or by supercritical CO₂, gamma, x-ray or electron beam irradiation.

In various embodiments, the injectable compositions of the present disclosure may contain one or more agents in addition to one or more types of iodinated polypeptide-containing molecules, the one or more types of silicate microparticles, and the water. Examples of such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the injectable compositions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

The injectable compositions of the present disclosure may be stored and transported in a sterile form. The injectable compositions may be shipped, for example, in a syringe, catheter, vial, ampoule, or other container.

In various embodiments, kits are provided, which may include one or more containers of injectable compositions as described herein as well other components. For example, the kits may include one or more delivery devices for delivering the injectable compositions to a subject such as syringes, catheters or tubing sets. In some embodiments, the kits may comprise an injectable composition as described herein preloaded in a catheter and/or a syringe barrel and/or in a container such as a vial or ampule. Alternatively or in addition, kits may be provided that include one or more accessory devices such as guidewires. Alternatively or in addition, the kits may be provided that include one or more containers of liquid materials (e.g. contrast agent, sterile water for injection, physiological saline, phosphate buffer, etc.). Alternatively or in addition, the kits may further comprise an additional therapeutic agent, which may be selected, for example, from those described above, among others. Instructions, either as inserts or as labels, indicating quantities of the composition to be administered and/or guidelines for administration can also be included in the kits provided herein. In some embodiments, the instructions comprise instructions for performing one or more of the methods provided herein.

The injectable compositions described herein can be administered by a variety of routes, depending upon the desired medical outcome. In some embodiments, the administering comprises injecting the injectable composition. In some embodiments, the injectable compositions are administered by parenteral administration. Parenteral administration includes intravenous, intraarterial, subcutaneous, intraperitoneal, or intramuscular injection or infusion. In some embodiments, the administering comprises an image guided procedure to deliver the composition. In some embodiments, the administering comprises injecting the injectable composition into the vascular system of a subject. In some embodiments, the administering comprises injecting the injectable composition into a cancer of the subject or the vasculature supplying a cancer of the subject. In some embodiments, the administering is performed using a catheter or a syringe.

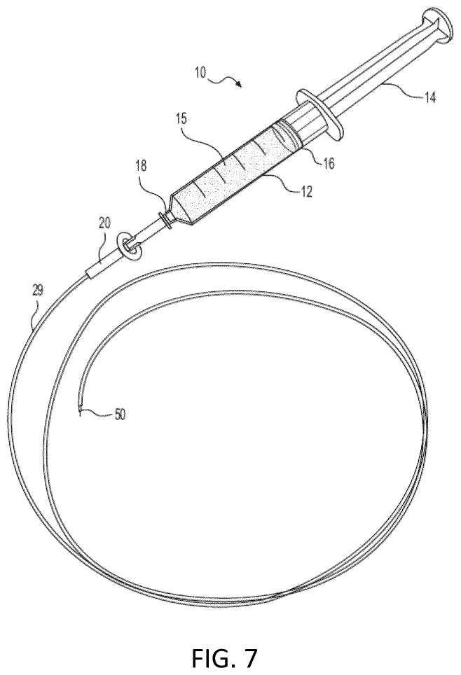

FIG. 7 illustrates an exemplary syringe 10 providing a reservoir for an injectable composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing an injectable composition 15 for injection through the needle 50.

The injectable compositions described herein can be administered to patients for achieving a number of medical outcomes.

The injectable compositions described herein can be visualized (e.g., within a mammal) using any appropriate method during and/or after administration. For example, the injectable compositions may be visualized using an X-ray based imaging technique, such as computerized tomography or X-ray fluoroscopy. Other imaging techniques such as ultrasound, magnetic resonance imaging, and/or fluoroscopy may also be used to visualize the injectable compositions provided herein.

The injectable compositions can be injected to provide spacing between tissues, injectable compositions can be injected (e.g., in the form of blebs) to provide fiducial markers, the injectable compositions can be injected for tissue augmentation or regeneration, injectable compositions can be injected as a filler or replacement for soft tissue, injectable compositions can be injected to provide mechanical support for compromised tissue, the injectable compositions can be injected as a scaffold, the injectable compositions can be injected as an embolic composition, the injectable compositions can be injected as lifting agents for internal cyst removal, and/or the injectable compositions can be injected as a carrier of therapeutic agents in the treatment of diseases and cancers and the repair and regeneration of tissue, among other uses. The injectable compositions can also be injected into a left atrial appendage during a left atrial appendage closure procedure. In some embodiments, the injectable compositions may be injected into the left atrial appendage after the introduction of a closure device such as the Watchman® left atrial appendage closure device available from Boston Scientific Corporation.

The injectable compositions of the present disclosure may be used in a variety of medical procedures, including the following, among others: a procedure to implant a fiducial marker comprising the injectable compositions, a procedure to implant a tissue regeneration scaffold comprising the injectable compositions, a procedure to implant a tissue support comprising the injectable compositions, a procedure to implant a tissue bulking agent comprising the injectable compositions, a procedure to implant a therapeutic-agent-containing depot comprising the injectable compositions, a tissue augmentation procedure comprising implanting the injectable compositions, a procedure to embolize tissue, including benign tumors, malignant tumors and other abnormal tissue, a procedure to control bleeding, a procedure to introduce the injectable compositions between a first tissue and a second tissue to space the first tissue from the second tissue.

The injectable compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable compositions may be injected for the permanent or temporary occlusion of blood vessels, and thus may be useful for managing various diseases and conditions. For example, the injectable compositions may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the particulate composition.

Injectable compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus, dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Injectable compositions in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

Although various embodiments are specifically illustrated and described herein, it will be appreciated that modifications and variations of the present disclosure are covered by the above teachings and are within the purview of any appended claims without departing from the spirit and intended scope of the present disclosure.

1. An injectable composition comprising (a) iodinated polypeptide-containing molecules, (b) silicate microparticles, and (c) water.

2. The injectable composition of claim 1, wherein the iodinated polypeptide-containing molecules are iodinated collagen-based proteins.

3. The injectable composition of claim 1, wherein the iodinated polypeptide-containing molecules comprise iodinated gelatin molecules.

4. The injectable composition of claim 1, wherein the silicate microparticles comprise natural and/or synthetic silicate layered clays.

5. The injectable composition of claim 1, wherein the injectable composition is an injectable shear-thinning composition.

6. The injectable composition of claim 1, wherein the iodinated polypeptide-containing molecules comprise covalently attached iodinated moieties.

7. The injectable composition of claim 6, wherein the iodinated polypeptide-containing molecules are formed by reacting amine groups of non-iodinated polypeptide-containing molecules with activated-ester-substituted iodinated molecules in an amide coupling process.

8. The injectable composition of claim 6, wherein the iodinated polypeptide-containing molecules are formed by (a) esterifying carboxyl groups of non-iodinated polypeptide-containing molecules to form esterified non-iodinated polypeptide-containing molecules and (b) reacting amine groups of the esterified non-iodinated polypeptide-containing molecules with carboxyl-substituted iodinated molecules in an amide coupling process.

9. The injectable composition of claim 8, wherein the iodinated polypeptide-containing molecules are de-esterified after the amide coupling process.

10. The injectable composition of claim 6, wherein the iodinated polypeptide-containing molecules are formed by reacting carboxyl groups of non-iodinated polypeptide-containing molecules with diazo-substituted iodinated molecules in an ester coupling process.

11. The injectable composition of claim 1, wherein the iodinated polypeptide-containing molecules comprise ionically coordinated carboxyl-substituted iodinated molecules.

12. The injectable composition of claim 1, wherein the injectable composition further comprises one or more additional agents selected from therapeutic agents, imaging agents, colorants, tonicity adjusting agents, suspension agents, wetting agents, and pH adjusting agents.

13. The injectable composition of claim 1, wherein the injectable composition is a sterile composition.

14. The injectable composition of claim 1, wherein the injectable composition is provided in a preloaded syringe.

15. A kit comprising one or more containers that a delivery device and an injectable composition comprising (a) iodinated polypeptide-containing molecules, (b) silicate microparticles, and (c) water.

16. The kit of claim 15, wherein the delivery device comprises a syringe, a needle and optionally, a catheter.

17. A medical procedure comprising administering an injectable composition comprising (a) iodinated polypeptide-containing molecules, (b) silicate microparticles, and (c) water to a subject.

18. The medical procedure of claim 17, wherein the method comprises injecting the injectable composition into the subject.

19. The medical procedure of claim 18, wherein the administering comprises parenteral administration.

20. The medical procedure of claim 18, wherein the administering is performed under image guidance.
